(19) United States
(12) Patent Application Publication
(10) Pub. No.: US 2024/0138721 A1
(43) Pub. Date: May 2, 2024

(54) APPARATUS AND METHOD FOR ESTIMATING CONCENTRATION OF ANALYTE COMPONENT

(71) Applicant: SAMSUNG ELECTRONICS CO., LTD., Suwon-si (KR)

(72) Inventors: Kun Sun EOM, Suwon-si (KR); JIN YOUNG PARK, Suwon-si (KR); MYOUNG HOON JUNG, Suwon-si (KR); YOON JAE KIM, Suwon-si (KR); HYUN SEOK MOON, Suwon-si (KR); JEONG EUN HWANG, Suwon-si (KR)

(73) Assignee: SAMSUNG ELECTRONICS CO., LTD., Suwon-si (KR)

(21) Appl. No.: 18/112,913

(22) Filed: Feb. 22, 2023

(30) Foreign Application Priority Data
Oct. 27, 2022 (KR) 10-2022-0140161

(51) Int. Cl. A61B 5/145; A61B 5/00; A61B 5/1455

(52) U.S. Cl. CPC A61B 5/14546; A61B 5/1455; A61B 5/684

(57) ABSTRACT

An apparatus for estimating a concentration of a component includes a sensor including a plurality of light sources having different central wavelengths and at least one detector configured to detect light, and a processor configured to determine a first reflectance of skin using a first light source of the plurality of light sources, set an operating condition of the sensor based on the determined first reflectance, drive the plurality of light sources according to the operating condition, and estimate a concentration of an analyte component based on a plurality of light quantities detected from skin by the at least one detector.

### CROSS-REFERENCE TO RELATED APPLICATION(S)

This application is based on and claims priority from Korean Patent Application No. 10-2022-0140161, filed on Oct. 27, 2022, in the Korean Intellectual Property Office, the disclosure of which is incorporated herein by reference in its entirety.

### BACKGROUND

### 1. Field

The present disclosure relates to an apparatus and method for estimating the concentration of an analyte component, and more particularly to a technology for dynamically setting a sensor operating condition based on a change in skin.

### 2. Description of the Related Art

Reactive oxygen species act as an important biological defense factor such as white blood cells protecting the body against infections. However, it has been known that excessive generation of reactive oxygen species in the body may lead to various tissue diseases. Common factors that cause the reactive oxygen species include stress, alcohol, peroxides, medicine, and the like. The reactive oxygen species produced by these factors may cause cranial nerve diseases, circulatory diseases, cancer, digestive tract diseases, liver diseases, arteriosclerosis, renal diseases, diabetes, aging, and the like. Human bodies have a series of antioxidant defense systems to protect against oxygen toxicity. For normal operation of the systems, it is essential to consume sufficient antioxidants such as vitamin E, vitamin C, carotenoid, flavonoid, etc., and it is important to eat as many foods that are rich in antioxidants as possible for an effective antioxidant action. Accordingly, there is a need for an apparatus for easily identifying the amount of antioxidant components in the body.

### SUMMARY

Provided are systems, methods and devices for accurately estimating a concentration of an antioxidant component by dynamically setting an operating condition of a sensor for estimating the concentration of the antioxidant component based on a change in skin.

Additional aspects will be set forth in part in the description which follows and, in part, will be apparent from the description, or may be learned by practice of the presented embodiments.

According to an aspect of the disclosure, an apparatus for estimating a concentration of a component may include a sensor including a plurality of light sources having different central wavelengths and at least one detector configured to detect light, and a processor configured to determine a first reflectance of skin using a first light source of the plurality of light sources, set an operating condition of the sensor based on the determined first reflectance, drive the plurality of light sources according to the operating condition, and estimate a concentration of an analyte component based on a plurality of light quantities detected from skin by the at least one detector.

The first light source of the plurality of light sources may have a central wavelength in a first range from about 350 nm to about 450 nm or in a second range from about 500 nm to about 600 nm.

The processor may be further configured to set the sensor to a first operating condition, drive the first light source based on the first operating condition, determine the first reflectance based on a first light quantity detected from the skin by the at least one detector, and based on the determined first reflectance being less than a first threshold value, change the operating condition to a second operating condition.

The processor may be further configured to determine the first reflectance based on an initial light quantity and the first light quantity, where wherein the initial light quantity is detected from a standard reflector using the first light source based on the first operating condition.

The processor may be further configured to determine, as the first reflectance, a result obtained by dividing the first light quantity by the initial light quantity.

The processor may be further configured to, based on the first reflectance being greater than or equal to the first threshold value, drive a second light source of the plurality of light sources and determine a second reflectance based on a second light quantity detected from the skin by the at least one detector, and, based on the determined second reflectance being greater than a second threshold value, change the operating condition to a third operating condition.

The second light source may have a central wavelength ranging from about 450 nm to about 500 nm.

The processor may be further configured to obtain a skin spectrum based on the plurality of light quantities, determine absorbance based on the obtained skin spectrum, and estimate the concentration of the analyte component based on the determined absorbance.

The processor may be further configured to, based on a predetermined condition being satisfied, determine an operating condition of the sensor.

The operating condition of the sensor may include a drive current for driving the plurality of light sources.

The processor may be further configured to set the sensor to a first operating condition, drive the first light source based on the first operating condition, determine a plurality of reflectances based on the plurality of light quantities detected from the skin by the at least one detector, and guide a user to change a position of the sensor based on the determined plurality of reflectances.

The analyte component may include at least one of skin carotenoid, blood carotenoid, glucose, urea, lactate, triglyceride, total protein, cholesterol, and ethanol.

According to an aspect of the disclosure, a method of estimating a concentration of a component may include determining a first reflectance of skin using a first light source of a plurality of light sources, the plurality of light sources being included in a sensor and having different central wavelengths, setting an operating condition of the sensor based on the determined first reflectance, driving the plurality of light sources based on the set operating condition, and estimating a concentration of an analyte component based on a plurality of light quantities detected from the skin by at least one detector included in the sensor.

The first light source may have a central wavelength in a first range from about 350 nm to about 450 nm or in a second range from about 500 nm to about 600 nm.

The setting of the operating condition of the sensor may include setting the sensor to a first operating condition, driving the first light source based on the first operating condition, determining the first reflectance based on a first light quantity detected from the skin by the at least one detector, and based on the determined first reflectance being less than a first threshold value, changing the operating condition to a second operating condition.

The determining of the first reflectance may include determining the first reflectance based on an initial light quantity and the first light quantity, where the initial light quantity is detected from a standard reflector using the first light source based on the first operating condition.

The setting of the operating condition of the sensor may include based on the first reflectance being greater than or equal to the first threshold value, driving a second light source of the plurality of light sources, determining a second reflectance based on a second light quantity detected from the skin by the at least one detector, and based on the determined second reflectance being greater than a second threshold value, changing the operating condition to a third operating condition.

The estimating of the concentration of the analyte component may include obtaining a skin spectrum based on the plurality of light quantities, determining absorbance using the obtained skin spectrum, and estimating the concentration of the analyte component based on the determined absorbance.

According to an aspect of the disclosure, a wearable device may include a main body, a sensor including a plurality of light sources having different central wavelengths and a detector configured to detect light from skin of the body of the user, and a processor configured to drive the plurality of light sources based on an operating condition of the sensor, estimate a concentration of an antioxidant component based on light detected by the detector, and based on a predetermined condition being satisfied, determine whether there is a change in color of the skin using a light source of the plurality of light sources having a predetermined central wavelength and set an operating condition of the sensor based on the determination of whether there is a change in the color of the skin.

The predetermined condition may be set in advance based on at least one of a period of time when the main body is not worn on the body of the user before the antioxidant component is estimated, a period of time during a day when the main body is not worn on the body of the user, and a season.

### BRIEF DESCRIPTION OF DRAWINGS

The above and other aspects, features, and advantages of certain embodiments of the present disclosure will be more apparent from the following description taken in conjunction with the accompanying drawings, in which:

FIG. 1 is a block diagram illustrating an apparatus for estimating a concentration of a component according to an embodiment of the present disclosure;

FIG. 2 is a diagram illustrating an example of a sensor structure of an apparatus for estimating a concentration of a component according to an embodiment of the present disclosure;

FIG. 3A is a diagram showing light absorption by blood components according to an embodiment of the present disclosure;

FIGS. 3B, 3C and 3D are diagrams of an example of obtaining an absorption spectrum according to an embodiment of the present disclosure;

FIG. 4 is a block diagram illustrating an apparatus for estimating a concentration of a component according to an embodiment of the present disclosure;

FIG. 5 is a diagram illustrating an example of guiding a sensor measurement position according to an embodiment of the present disclosure;

FIG. 6 is a flowchart of a method of estimating a concentration of an analyte component according to an embodiment of the present disclosure;

FIGS. 7A, 7B and 7C are flowcharts of methods of setting an operating condition based on a change in skin according to an embodiment of the present disclosure; and

FIGS. 8, 9 and 10 are diagrams illustrating examples of structures of an electronic device including an apparatus for estimating a concentration of a component according to an embodiment of the present disclosure.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

Details of other embodiments are included in the following detailed description and drawings. Advantages and features of the present invention, and a method of achieving the same will be more clearly understood from the following embodiments described in detail with reference to the accompanying drawings. Throughout the drawings and the detailed description, unless otherwise described, the same drawing reference numerals will be understood to refer to the same elements, features, and structures.

It will be understood that, although the terms first, second, etc. may be used herein to describe various elements, these elements should not be limited by these terms. These terms are only used to distinguish one element from another. Any references to singular may include plural unless expressly stated otherwise. In addition, unless explicitly described to the contrary, an expression such as “comprising” or “including” will be understood to imply the inclusion of stated elements but not the exclusion of any other elements. Also, the terms, such as ‘unit’ or ‘module’, etc., should be understood as a unit that performs at least one function or operation and that may be embodied as hardware, software, or a combination thereof.

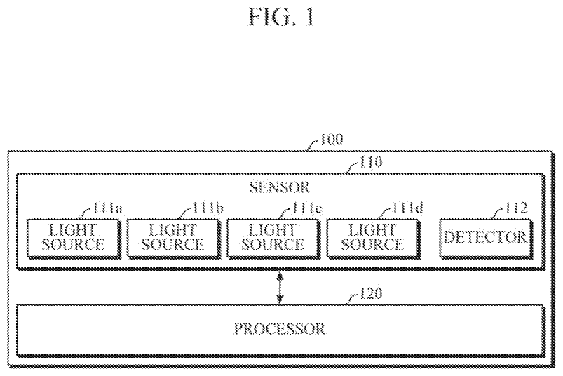

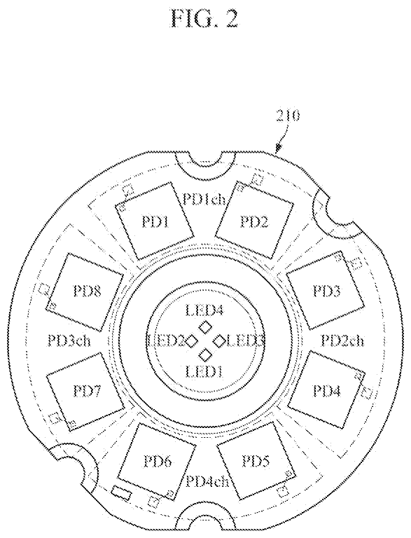

FIG. 1 is a block diagram illustrating an apparatus for estimating a concentration of an analyte component according to an embodiment of the present disclosure. FIG. 2 is a diagram illustrating an example of a sensor structure of an apparatus for estimating a concentration of a component according to an embodiment of the present disclosure.

Referring to FIG. 1, an apparatus 100 for estimating a concentration of a component includes a sensor 110 and a processor 120.

The sensor 110 may include a plurality of light sources 111a, 111b, 111c, and 111d. While four light sources 111a, 111b, 111c, and 111d are illustrated for convenience of explanation, the number of light sources is not limited thereto. Each of the light sources 111a, 111b, 111c, and 111d may be a light emitting diode (LED), a laser diode, a phosphor, etc., but the light sources are not limited to these examples. The plurality of light sources 111a, 111b, 111c, and 111d may be driven simultaneously or in a time-division manner. Alternatively, the light sources may be driven in a generally sequential manner with predetermined light sources being simultaneously driven in groups.

The respective light sources 111a, 111b, 111c, and 111d may have different central wavelengths. For example, a first light source 111a may have a central wavelength ranging from about 350 nm to about 450 nm, and a second light source 111b may have a central wavelength ranging from about 450 nm to about 500 nm. In addition, a third light source 111c and a fourth light source 111d may have central wavelengths ranging from about 500 nm to about 550 nm and ranging from about 550 nm to about 650 nm, respectively. However, the central wavelengths of the light sources are not limited thereto and may be changed according to various conditions, such as the type of analyte component, the size of a form factor of the apparatus 100, and the like.

The sensor 110 may include a detector 112 configured to detect light, convert the light into an electrical signal, and output the electrical signal. The detector 112 may include one or more photo diodes, photo transistors (PTr), etc., but is not limited thereto, and may include a complementary metal oxide semiconductor (CMOS) image sensor, a charge-coupled device (CCD) image sensor, and the like.

The sensor 110 may include an analog-digital converter configured to convert an electrical signal, output from the detector 112, into a digital signal and/or an amplifier configured to amplify the electrical signal.

Referring to FIG. 2, the sensor 110 according to an embodiment has a structure in which a plurality of LEDs (LED1 to LED4) having different central wavelengths are disposed at the center, and a plurality of photodiodes PD1 to PD8 are disposed around the periphery thereof. However, the sensor 110 is not limited thereto, and one or more photodiodes may be disposed at the center, and the plurality of LEDs (LED1 to LED4) may be disposed around the periphery thereof. In this case, each one of channels PD1ch to PD4ch include two or more of the photodiodes PD1 to PD8, such that the photodiodes may be driven on a channel basis (e.g., PD1 and PD2 may be driven on PD1ch, PD3 and PD4 may be driven on PD2ch, PD5 and PD6 may be driven on PD4ch, and PD7 and PD8 may be driven on PD3ch). The photodiodes PD1 to PD8 may be driven in synchronization with the light sources which are driven individually or on a channel basis. For example, LED1 may be synchronized with PD1ch, LED2 may be synchronized with PD2ch, LED3 may be synchronized with PD3ch, and LED4 may be synchronized with PD4ch.

Referring back to FIG. 1, the processor 120 may be electrically connected to the sensor 110. The processor 120 may set an operating condition of the sensor 110 and may control the sensor 110 according to the set operating condition. In this case, the operating condition may include parameters, such as magnitude of incident light (e.g., current intensity), gain, exposure time, aperture size, and the like. A plurality of operating conditions may be predefined by combining the parameter values, and when driving the sensor 110, the processor 120 may set one of the operating conditions as the operating condition of the sensor 110. The plurality of operating conditions may include an initial operating condition defined to be universally applied regardless of skin color, and various operating conditions defined in consideration of skin color of ordinary people or a change in skin color of an individual user.

In this case, a user's skin is a body part coming into contact with the sensor 110, and may be, for example, the palm of the hand or the sole of the foot which is covered with a thick epidermis layer, a body part that is adjacent to the radial artery or where venous blood or capillary blood passes, a peripheral part of the body with high blood vessel density, such as fingers, toes, earlobes, or the like, or the wrist and an inner part of the ear which may come into contact with a wearable device when the device is worn, and the like.

Upon receiving a user's request for estimating a concentration of an analyte component, the processor 120 may operate the sensor 110 and may estimate the concentration of the analyte component by using data obtained by the sensor 110. In this case, the analyte component may include an antioxidant component including skin carotenoid and/or blood carotenoid, or a component such as glucose, urea, lactate, triglyceride, total protein, cholesterol, ethanol, etc., but is not limited thereto. For convenience of explanation, the following description will be given using an antioxidant component as an example.

Upon receiving a request for estimating a concentration of an antioxidant component, the processor 120 may determine a change in skin color of a user and may set an operating condition suitable for estimating the concentration of the antioxidant component based on the determination, before estimating the concentration. Generally, the skin color is associated with melanin distribution, and melanin affects light absorption, thereby affecting a signal quality of reflected light. Accordingly, by properly setting an operating condition for the sensor in consideration of a change in a user's skin color, the signal quality of light acquired by the sensor 110 may be improved.

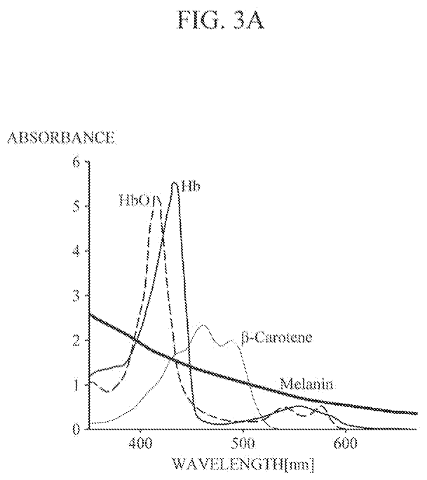

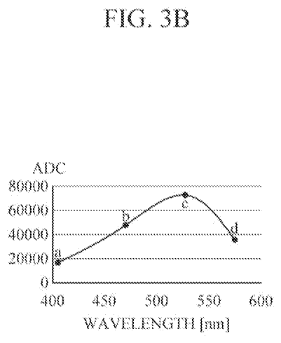

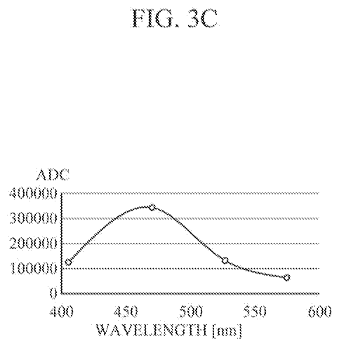

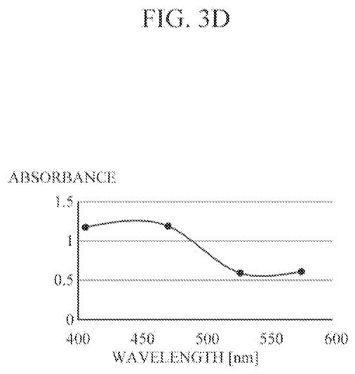

FIG. 3A is a diagram showing light absorption by blood components according to an embodiment of the present disclosure.

As illustrated herein, it can be seen that components, such as melanin, hemoglobin, and the like in blood, exhibit high light absorption in a relatively short-wavelength region (e.g., about 350 nm to about 450 nm). Particularly, light absorption by melanin gradually increases towards the short-wavelength region, such that a magnitude of reflected light in the short-wavelength region may affect a signal quality.

Accordingly, among the plurality of light sources 111a, 111b, 111c, and 111d included in the sensor 110, the processor 120 may drive the first light source 111a having a central wavelength (e.g., ranging from about 350 nm to about 450 nm according to a first operating condition) and may determine a change in skin color related to the concentration of melanin. In this case, the first operating condition may be preset by a user, and may be, for example, the aforementioned initial operating condition, a basic operating condition set by a user among a plurality of operating conditions, or an operating condition set at a most recent estimation time.

When the first light source 111a emits light to skin, and the detector 112 detects light scattered or reflected from the skin, the processor 120 may calculate a first reflectance based on a detected light quantity and may determine a change in skin color based on the calculated first reflectance. For example, the processor 120 may obtain, as the first reflectance, a result obtained by dividing the detected light quantity by an initial light quantity measured in advance for the first light source 111a according to the first operating condition. In this case, the initial light quantity may be measured in advance by using a standard reflector for each of the plurality of light sources 111a, 111b, 111c, and 111d according to each of a plurality of operating conditions. For example, the initial light quantity may be measured during the manufacture of the sensor 110 or during the first use of the apparatus 100. In this case, the standard reflector may be a reflector having a predetermined reflectance (e.g., average reflectance (about 50%) or maximum reflectance (about 80%) of human skin).

For example, the processor 120 may compare the calculated first reflectance with a predetermined first threshold value (e.g., about 7%). If the first reflectance is greater than or equal to the first threshold value, the processor 120 may determine that a change in skin color is not large, and may maintain the set first operating condition (e.g., drive current of 5 mA). If the first reflectance is less than the first threshold value, the processor 120 may determine that skin color is changed, and may change the operating condition of the sensor 110 from the first operating condition (e.g., drive current of 5 mA) to a second operating condition (e.g., drive current of 20 mA). In this case, by dividing a degree, by which the first reflectance is less than the first threshold value, into a plurality of levels, the processor 120 may change the operating condition according to each level. The drive current under each operating condition may be set differently for each light source.

In another example, if the calculated first reflectance is less than the first threshold value, the processor 120 may determine that skin color is changed, and may change the operating condition to the second operating condition; and if not, the processor 120 may determine a possibility of signal saturation. The processor 120 may drive the second light source 112b, having a central wavelength in a wavelength range (e.g., about 450 nm to about 500 nm) with a relatively large magnitude of skin reflected light, among the plurality of light sources 111a, 111b, 111c, and 111d according to the first operating condition, and may determine signal saturation by using a second light quantity detected by the detector. For example, the processor 120 may obtain a second reflectance by dividing the second light quantity by an initial light quantity measured for the second light source according to the first operating condition. If the second reflectance is greater than a predetermined second threshold value, the processor 120 may determine that there is a possibility of signal saturation, and may change the operating condition of the sensor 110 to a third operating condition (e.g., drive current of 3 mA), and if not, the processor 120 may maintain the first operating condition.

While an example of determining a change in skin color by using the light source having the central wavelength ranging from about 350 nm to about 450 nm is described above, the determination is not limited thereto, and if there is no light source having the central wavelength of about 350 nm to about 450 nm, a light source having a central wavelength of about 500 nm to about 600 nm may also be used. Alternatively, a change in skin color may be determined by using a plurality of light sources having central wavelengths (e.g., ranging from about 350 nm to about 450 nm and ranging from about 500 nm to about 600 nm). For example, the processor 120 may calculate the aforementioned first reflectance by using a statistical value (e.g., sum, average, etc.) of quantities of light detected from the plurality of light sources and a statistical value (e.g., sum, average, etc.) of initial light quantities corresponding thereto.

The aforementioned process of setting the sensor operating condition based on the change in skin color may be performed if a predetermined condition is satisfied. In this case, the predetermined condition may be set so that the setting of the sensor operating condition may be performed when the apparatus 110 is first used and if there is a user's request. Alternatively, if no measurement is performed after a given period of time has elapsed, there is a possibility that skin color may change with an increasing time of exposure of skin to the sun, such that measurement may be set to be performed when a given period of time elapses after a previous measurement time. In this case, the given period of time may be set differently according to gender, age, daytime and nighttime, and/or season, and the like.

In order to determine the change in skin color, the processor 120 may drive the first light source according to the first operating condition, and may guide a user to adjust a sensor measurement position based on a plurality of light quantities detected by the plurality of detectors. For example, upon determining, based on the respective light quantities detected by the plurality of detectors, that a change occurs in the entire area or most areas of skin coming into contact with the sensor 110, the processor 120 may change the operating condition of the sensor as described above. Upon determining that only a partial area of skin has a portion which is different in color (e.g., dark spot) or is changed, the processor 120 may guide a user to change a sensor position to another position.

For example, the processor 120 may calculate reflectances for each of the plurality of detectors based on light quantities detected by the respective detectors and the initial light quantity as described above, and may compare the respective reflectances with a threshold value. If a percentage of the number of reflectances, which are less than the threshold value, is a predetermined percentage or more with respect to the total number of reflectances, the processor 120 may determine that the entire area of skin is changed in color. If a percentage of the number of reflectances, which are less than the threshold value, is not a predetermined percentage or more with respect to the total number of reflectances, the processor 120 may determine that only a partial area of skin is changed in color, and may guide a user to change a measurement position in consideration of a position of a specific detector/detector channel corresponding to the reflectance which is less than the threshold value.

The processor 120 may drive the plurality of light sources 111a, 111b, 111c, and 111d included in the sensor 110 according to the operating condition, which is set based on the above determination on the change in skin color, or according to the first operating condition if the process of determining a change in skin color is omitted, and may estimate a concentration of an antioxidant component based on the plurality of light quantities detected by the detector 112. In this case, the first operating condition may be the initial operating condition, the basic operating condition, or the operating condition set at a previous estimation time, as described above.

FIGS. 3B, 3C and 3D are diagrams of an example of obtaining an absorption spectrum according to an embodiment of the present disclosure. FIG. 3B is a diagram illustrating a spectrum reconstructed based on light quantities a, b, c, and d measured using the light sources 111a, 111b, 111c, and 111d having difference central wavelengths, and FIG. 3C is a diagram illustrating a calibration spectrum obtained based on the initial light quantity measured for the respective light sources 111a, 111b, 111c, and 111d by using a standard reflector. FIG. 3D is a diagram illustrating an example of a skin absorption spectrum obtained by correcting the reconstructed spectrum of FIG. 3B using the calibration spectrum of FIG. 3C.

For example, when the plurality of light sources 111a, 111b, 111c, and 111d are driven under the set operating condition and a plurality of light quantities are detected, the processor 120 may reconstruct a skin spectrum based on the detected plurality of light quantities, and may obtain an absorption spectrum based on Lambert-Beer's law by using a calibration spectrum corresponding to the set operating condition. For example, if the operating condition of the sensor 110 is changed from the first operating condition to the second operating condition, the processor 120 may obtain a skin absorption spectrum by using the calibration spectrum obtained using the standard reflector according to the second operating condition.

Upon obtaining the skin absorption spectrum, the processor 120 may estimate a concentration of an antioxidant component by using the skin absorption spectrum. For example, the processor 120 may estimate a concentration of an analyte component by applying a predefined estimation model to the obtained absorbance. In this case, the estimation model may be defined in advance as, for example, linear combination equation, but is not limited thereto.

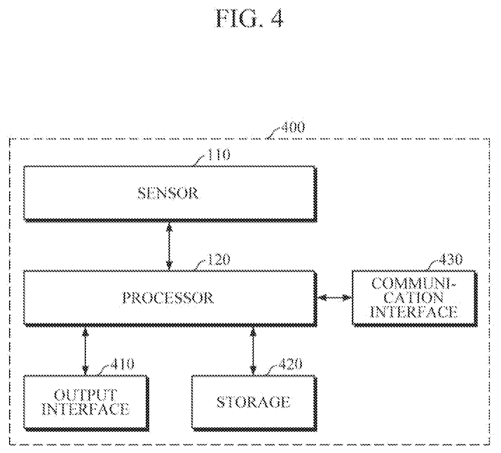

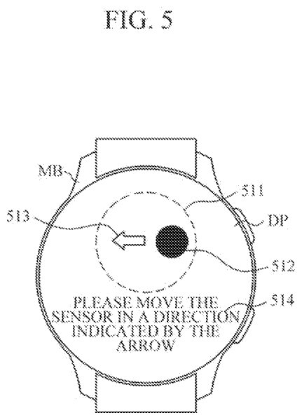

FIG. 4 is a block diagram illustrating an apparatus for estimating a concentration of a component according to an embodiment of the present disclosure. FIG. 5 is a diagram illustrating an example of guiding a sensor measurement position according to an embodiment of the present disclosure.

Referring to FIG. 4, an apparatus 400 for estimating a component includes the sensor 110, the processor 120, an output interface 410, a storage 420, and a communication interface 430. The sensor 110 may include a plurality of light sources and detectors. The sensor 110 and the processor 120 are described above, such that a detailed description thereof will be omitted.

The output interface 410 may output data processed by the processor 120. For example, the output interface 410 may output information, such as spectrum data, a result of estimating a concentration of an analyte component, sensor operating conditions, and the like. The output interface 410 may include a visual output module such as a display, an audio output module such as a speaker, or a haptic module using vibrations, tactile sensation, and the like. For example, the output interface 410 may output information, such as the spectrum data, analyte component concentration estimation data, sensor operating conditions, and the like.

In addition, the output interface 410 may output information for guiding a user to adjust a sensor measurement position generated by the processor 120. As illustrated in FIG. 5, the output interface 410 may output, to a display DP of a main body MB, a graphic object 511 (e.g., a circular box, a square box, skin image, etc.), which indicates a skin area 511 coming into contact with the sensor 110, an object 512 indicating a position at which skin color is different, a circular box or an image. In this case, the depth of the color of the object 512 may be set differently according to a degree of color change (e.g., degree of difference between a threshold value and reflectance). In addition, the output interface 410 may output an object 513, such as an arrow indicating a moving direction, and/or a text message 514, such as “please move the sensor in a direction indicated by the arrow,” in order to move a sensor position.

The storage 420 may store data related to estimating a component concentration. For example, the storage 420 may store user characteristic information, such as a user's age, gender, health condition, etc., various sensor operating conditions, initial light quantity, component estimation model, and the like. Further, the storage 420 may store information, such as an estimated component concentration value generated by the processor 120, and the like.

The storage 420 may include at least one storage medium of a flash memory type memory, a hard disk type memory, a multimedia card micro type memory, a card type memory (e.g., a secure digital (SD) memory, an XD memory, etc.), a random access memory (RAM), a static random access memory (SRAM), a read only memory (ROM), an electrically erasable programmable ROM (EEPROM), a programmable ROM (PROM), a magnetic memory, a magnetic disk, and an optical disk, and the like, but is not limited thereto.

The communication interface 430 may communicate with an external device by wired or wireless communications to receive various data related to estimating a component. The external device may include an information processing device such as a smartphone, a tablet PC, a laptop computer, a desktop computer, etc., but is not limited thereto. The communication interface 430 may communicate with the external device by using communication techniques, such as Bluetooth communication, Bluetooth low energy (BLE) communication, near field communication (NFC), wireless local access network (WLAN) communication, Zigbee communication, infrared data association (IrDA) communication, Wi-Fi direct (WFD) communication, ultra-wideband (UWB) communication, Ant+ communication, Wi-Fi communication, radio frequency identification (RFID) communication, 3^{rd} generation (3G), 4^{th} generation (4G), and 5^{th} generation (5G) communications, and the like. However, this is merely exemplary and is not intended to be limiting.

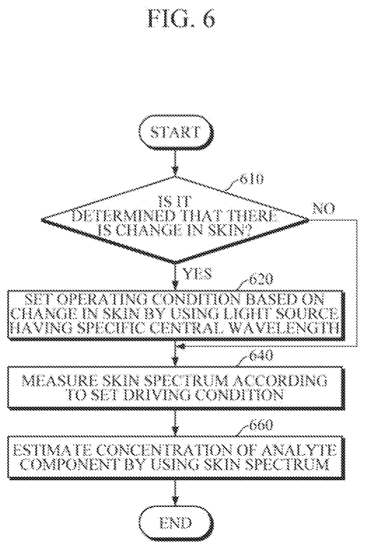

FIG. 6 is a flowchart of a method of estimating a concentration of an analyte component according to an embodiment of the present disclosure. The method of estimating a concentration of an analyte component may be performed by the aforementioned apparatuses 100 and 400 for estimating a concentration of a component, which are described in detail above, and thus will be briefly described below.

Referring to FIG. 6A, in response to a request for estimating a concentration of an analyte component, in operation 610, the apparatus for estimating a concentration of a component may determine whether there is a change in skin color by using one light source having a specific central wavelength among the plurality of light sources included in the sensor. Based on determining that there is no change in skin, the apparatus may proceed to operation 640. Based on determining that there is a change in skin, in operation 620, the apparatus may set a sensor operating condition suitable for estimating a component concentration. In this case, the plurality of light sources may have different central wavelengths.

In an embodiment, in operation 610 or prior to operation 610, in response to the request for estimating the concentration of the analyte component, the apparatus for estimating a concentration of a component may first determine whether a predetermined condition is satisfied prior to operation 610, and if the predetermined condition is satisfied, the apparatus for estimating a concentration of a component may determine whether there is a change in skin in operation 610 and may set a sensor operating condition based on the skin change in operation 620. In this case, the predetermined condition may be set such that the determining whether there is a change in skin may be performed at the first use of the apparatus, at predetermined intervals, or if measurement with the sensor is not performed after a predetermined period of time or more has elapsed.

Then, the apparatus for estimating a concentration of a component may measure a skin spectrum by driving the plurality of light sources and detectors according to the set sensor operating condition in operation 640. The apparatus for estimating a concentration of a component may reconstruct the spectrum by driving the plurality of light sources according to the set sensor operating condition and by using light quantities detected for the respective light sources, and may generate a skin absorption spectrum based on a calibration spectrum measured using a standard reflector according to the set sensor operating condition.

Subsequently, the apparatus for estimating a concentration of a component may estimate the concentration of the analyte component by using the skin spectrum in operation 660. For example, the apparatus for estimating a concentration of a component may estimate the concentration of the component by applying a predefined component estimation model to absorbances obtained for the respective light sources.

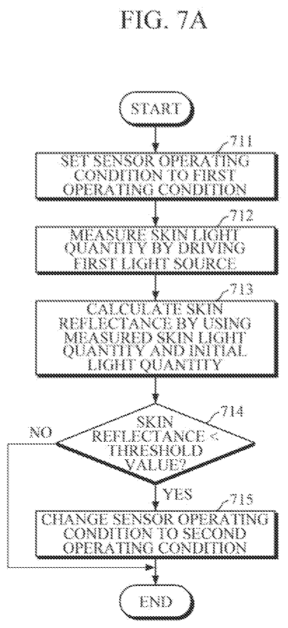

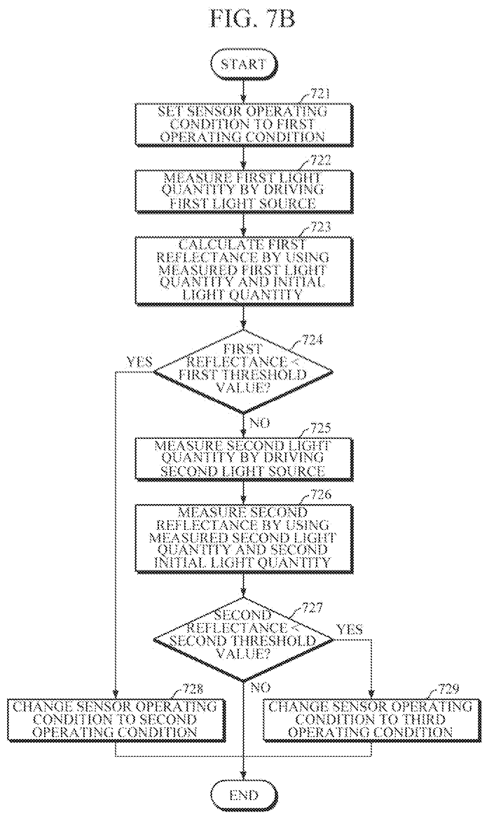

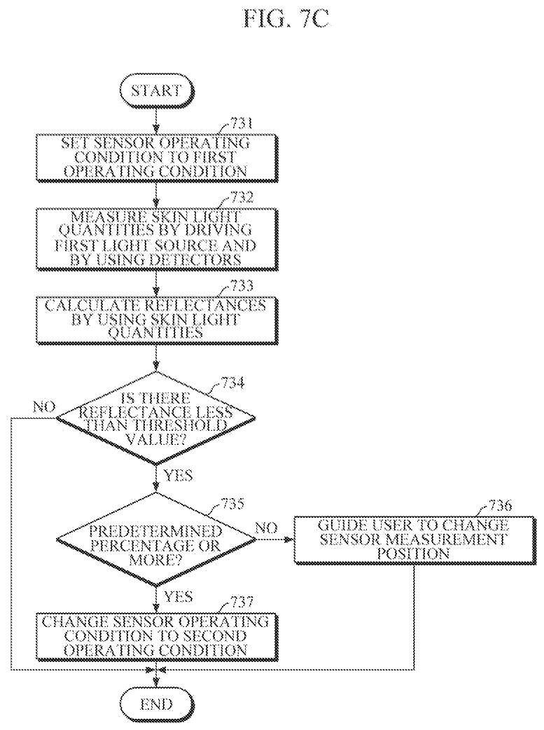

FIGS. 7A, 7B and 7C are flowcharts of methods of setting an operating condition based on a change in skin according to an embodiment of the present disclosure. FIGS. 7A to 7C are diagrams illustrating various examples of setting the sensor operating condition in 620 of FIG. 6.

Referring to FIG. 7A, the apparatus for estimating a concentration of a component may set the sensor operating condition to a first operating condition in operation 711. The first operating condition may be an initial operating condition, a basic operating condition set by a user, or an operating condition set at a previous estimation time, and may maintain an operating condition set at the previous measurement time according to, for example, a user's setting, or may reset the operating condition to the initial operating condition or the basic operating condition.

Then, by driving the first light source according to the set first operating condition, the apparatus for estimating a concentration of a component may measure a skin light quantity by using the detector in operation 712. In this case, the first light source may be a light source having a central wavelength of about 350 nm to about 450 nm. However, the light source is not limited thereto, and a light source having a central wavelength of about 500 nm to about 600 nm may also be used as needed, and a plurality of light sources having central wavelengths in a range of about 350 nm to about 450 nm and a range of about 500 nm to about 600 nm may also be used.

Subsequently, the apparatus for estimating a concentration of a component may calculate skin reflectance in operation 713 by using the light quantity measured in operation 712 and the initial light quantity. In this case, the initial light quantity may be a quantity of reflected light which is measured using a standard reflector by driving the first light source according to the first operating condition.

Next, the apparatus for estimating a concentration of a component may compare the calculated skin reflectance with a threshold value in operation 714. Upon comparison, if the skin reflectance is greater than or equal to the threshold value, the apparatus for estimating a concentration of a component may maintain the current first operating condition, and if the skin reflectance is less than the threshold value, the apparatus for estimating a concentration of a component may change the sensor operating condition to the second operating condition in operation 715.

Referring to FIG. 7B, the apparatus for estimating a concentration of a component may first set the sensor operating condition to the first operating condition in operation 721. The first operating condition may be an initial operating condition, a basic operating condition set by a user, or an operating condition set at a previous estimation time.

Then, by driving the first light source according to the set first operating condition, the apparatus for estimating a concentration of a component may measure a first light quantity by using the detector in operation 722. In this case, the first light source may be a light source having a central wavelength of about 350 nm to about 450 nm. However, the light source is not limited thereto, and a light source having a central wavelength of about 500 nm to about 600 nm may also be used as needed, and a plurality of light sources having central wavelengths in a range of about 350 nm to about 450 nm and a range of about 500 nm to about 600 nm may also be used.

Subsequently, the apparatus for estimating a concentration of a component may calculate a first reflectance in operation 723 by using the first light quantity measured in operation 722 and the initial light quantity. In this case, the initial light quantity may be a quantity of reflected light which is measured using a standard reflector by driving the first light source according to the first operating condition.

Next, the apparatus for estimating a concentration of a component may compare the calculated first reflectance with a first threshold value in operation 724. Upon comparison, if the first reflectance is less than the threshold value, the apparatus for estimating a concentration of a component may change the sensor operating condition to the second operating condition in operation 728. Upon comparison, if the first reflectance is greater than or equal to the threshold value, the apparatus for estimating a concentration of a component may drive the second light source to measure a second light quantity in operation 725. In this case, the second light source may be a light source having a central wavelength of about 450 nm to about 500 nm.

Then, the apparatus for estimating a concentration of a component may measure a second reflectance by using the measured second light quantity and a second initial light quantity in operation 726. In this case, the second initial light quantity may be a quantity of reflected light which is measured using a standard reflector by driving the second light source according to the first operating condition.

Subsequently, the apparatus for estimating a concentration of a component may compare the second reflectance with a second threshold value in operation 727, and if the second reflectance is less than or equal to the second threshold value, the apparatus for estimating a concentration of a component may maintain the current first operating condition, and if the second reflectance is greater than the second threshold value, the apparatus for estimating a concentration of a component may change the sensor operating condition to a third operating condition in operation 729.

Referring to FIG. 7C, the apparatus for estimating a concentration of a component may set the sensor operating condition to the first operating condition in operation 731.

Then, the apparatus for estimating a concentration of a component may measure a plurality of skin light quantities by driving the first light source according to the first operating condition and by using the plurality of detectors in operation 732.

Subsequently, the apparatus for estimating a concentration of a component may calculate skin reflectances in operation 733 for each of the detectors by using the light quantities measured in operation 732 by the respective detectors and the initial light quantity.

Next, the apparatus for estimating a concentration of a component may compare the skin reflectances, calculated for each of the detectors, with a threshold value and may determine whether there is a reflectance less than the threshold value in operation 734. Upon comparison, if all the reflectances are greater than or equal to the threshold value, the apparatus for estimating a concentration of a component may maintain the current first operating condition.

Upon comparison, if there is a reflectance less than the threshold value, the apparatus for estimating a concentration of a component may determine whether a percentage of the number of reflectances, which are less than the threshold value, is a predetermined percentage or more with respect to the total number of reflectances in operation 735. If the percentage of the number of reflectances is not the predetermined percentage or more with respect to the total number of reflectances, the apparatus for estimating a concentration of a component may determine that only a partial area of skin is changed in color and may guide a user to change a sensor measurement position in operation 736. If the percentage of the number of reflectances, which are less than the threshold value, is the predetermined percentage or more with respect to the total number of reflectances, the apparatus for estimating a concentration of a component may determine that the entire area of skin is changed in color and may change the sensor operating condition to the second operating condition in operation 737.

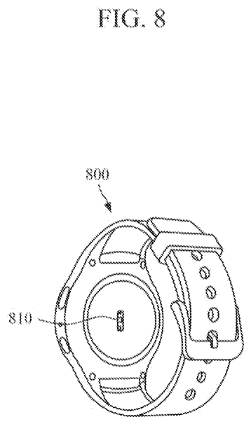

FIGS. 8, 9 and 10 are diagrams illustrating examples of structures of an electronic device including an apparatus for estimating a concentration of a component according to an embodiment of the present disclosure. FIGS. 8 to 10 are diagrams illustrating examples of various structures of an electronic device including the apparatuses 100 and 400 for estimating a concentration of a component.

The electronic device may include, for example, various types of wearable devices (e.g., a smart watch, a smart band, smart glasses, smart earphones, a smart ring, a smart patch, and a smart necklace, and a mobile device such as a smartphone, a tablet PC, etc.), or home appliances or various Internet of Things (IoT) devices (e.g., home IoT device, etc.) based on IoT technology.

The electronic device may include a sensor device, a processor, an input device, a communication module, a camera module, an output device, a storage device, and a power module. All the components of the electronic device may be integrally mounted in a specific device or may be distributed in two or more devices. The sensor device may include the sensor 110 of the apparatuses 100 and 400 for estimating a concentration of a component, and may further include an additional sensor, such as a gyro sensor, a global positioning system (GPS), and the like.

The processor may execute programs, stored in the storage device, to control components connected to the processor, and may perform various data processing or computation, including estimation of bio-information. The processor may include a main processor (e.g., a central processing unit (CPU) or an application processor (AP), etc.), and an auxiliary processor (e.g., a graphics processing unit (GPU), an image signal processor (ISP), a sensor hub processor, or a communication processor (CP), etc.), which is operable independently from, or in conjunction with, the main processor.

The input device may receive a command and/or data to be used by each component of the electronic device, from a user and the like. The input device may include, for example, a microphone, a mouse, a keyboard, or a digital pen (e.g., a stylus pen, etc.).

The communication module may support establishment of a direct (e.g., wired) communication channel and/or a wireless communication channel between the electronic device and other electronic device, a server, or the sensor device within a network environment, and performing of communication via the established communication channel. The communication module may include one or more communication processors that are operable independently from the processor and supports a direct communication and/or a wireless communication. The communication module may include a wireless communication module (e.g., a cellular communication module, a short-range wireless communication module, or a global navigation satellite system (GNSS) communication module, etc.), and/or a wired communication module (e.g., a local area network (LAN) communication module, a power line communication (PLC) module, and the like). These various types of communication modules may be integrated into a single chip, or may be separately implemented as multiple chips. The wireless communication module may identify and authenticate the electronic device in a communication network by using subscriber information (e.g., international mobile subscriber identity (IMSI), etc.) stored in a subscriber identification module.

The camera module may capture still images or moving images. The camera module may include a lens assembly having one or more lenses, image sensors, image signal processors, and/or flashes. The lens assembly included in the camera module may collect light emanating from a subject to be imaged.

The output device may visually/non-visually output data generated or processed by the electronic device. The output device may include a sound output device, a display device, an audio module, and/or a haptic module.

The sound output device may output sound signals to the outside of the electronic device. The sound output device may include a speaker and/or a receiver. The speaker may be used for general purposes, such as playing multimedia or playing record, and the receiver may be used for incoming calls. The receiver may be implemented separately from, or as part of, the speaker.

The display device may visually provide information to the outside of the electronic device. The display device may include, for example, a display, a hologram device, or a projector and control circuitry to control the devices. The display device may include touch circuitry adapted to detect a touch, and/or sensor circuitry (e.g., pressure sensor, etc.) adapted to measure the intensity of force incurred by the touch.

The audio module may convert a sound into an electrical signal or vice versa. The audio module may obtain the sound via the input device, or may output the sound via the sound output device, and/or a speaker and/or a headphone of another electronic device directly or wirelessly connected to the electronic device.

The haptic module may convert an electrical signal into a mechanical stimulus (e.g., vibration, motion, etc.) or electrical stimulus which may be recognized by a user by tactile sensation or kinesthetic sensation. The haptic module may include, for example, a motor, a piezoelectric element, and/or an electric stimulator.

The storage device may store operating conditions required for operating the sensor device, and various data necessary for other components of the electronic device. The various data may include, for example, input data and/or output data for software and instructions associated with the software, and the like. The storage device may include a volatile memory and/or a non-volatile memory.

The power module may manage power supplied to the electronic device. The power module may be implemented as part of, for example, a power management integrated circuit (PMIC). The power module may include a battery, which may include a primary cell which is not rechargeable, a secondary cell which is rechargeable, and/or a fuel cell.

Referring to FIG. 8, the electronic device may be implemented as a wristwatch wearable device 800, and may include a main body and a wrist strap. A display is provided on a front surface of the main body, and may display various application screens, including an estimated analyte component concentration value, warning information, time information, received message information, and the like. A sensor device 810 may be disposed on a rear surface of the main body. During estimation of a concentration of an analyte component, the wearable device 800 may first determine whether there is a change in skin color and may basically perform an operation of resetting a sensor operating condition based on the determination, or may check a predetermined condition based on a period of time when the wearable device 800 is not worn on the wrist, season, daytime or nighttime, etc., and if the predetermined condition is satisfied, the wearable device 800 may perform the operation of resetting the sensor operating condition.

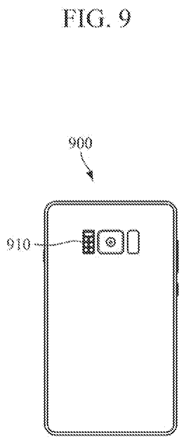

Referring to FIG. 9, the electronic device may be implemented as a mobile device 900 such as a smartphone. The mobile device 900 may include a housing and a display panel. The housing may form an exterior of the mobile device 900. The housing has a first surface, on which a display panel and a cover glass may be disposed sequentially, and the display panel may be exposed to the outside through the cover glass. A sensor device 910, a camera module and/or an infrared sensor, and the like may be disposed on a second surface of the housing. The processor and various other components may be disposed in the housing.

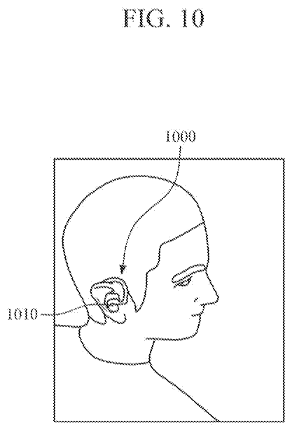

Referring to FIG. 10, the electronic device may be implemented as an ear-wearable device 1000. The ear-wearable device 1000 may include a main body and an ear strap. A user may wear the ear-wearable device 1000 by hanging the ear strap on the auricle. The ear strap may be omitted depending on a shape of the ear-wearable device 1000. The main body may be inserted into the external auditory meatus. A sensor device 1010 may be mounted in the main body at a portion coming into contact with skin. Further, a processor, a communication device, and the like may be mounted in the main body. The processor may estimate a concentration of a component based on light measured by the sensor 1010 as described above, and may transmit an estimation result to another electronic device through the communication device. Alternatively, the processor may transmit light data, measured by the sensor 1010, to another electronic device so that the electronic device may estimate the component concentration.

The present disclosure can be realized as a computer-readable code written on a computer-readable recording medium. The computer-readable recording medium may be any type of recording device in which data is stored in a computer-readable manner.

Examples of the computer-readable recording medium include a ROM, a RAM, a compact disc (CD) ROM (CD-ROM), a magnetic tape, a floppy disc, an optical data storage, and a carrier wave (e.g., data transmission through the Internet). The computer-readable recording medium can be distributed over a plurality of computer systems connected to a network so that a computer-readable code is written thereto and executed therefrom in a decentralized manner. Functional programs, codes, and code segments needed for realizing the present invention can be readily deduced by programmers of ordinary skill in the art to which the invention pertains.

The disclosure has been described herein with regard to preferred embodiments. However, it will be obvious to those skilled in the art that various changes and modifications can be made without changing technical conception and essential features of the present disclosure. Thus, it is clear that the above-described embodiments are illustrative in all aspects and are not intended to limit the present disclosure.

What is claimed is:

1. An apparatus for estimating a concentration of a component, the apparatus comprising:
a sensor comprising:
a plurality of light sources having different central wavelengths; and
at least one detector configured to detect light; and
a processor configured to:
determine a first reflectance of skin using a first light source of the plurality of light sources,
set an operating condition of the sensor based on the determined first reflectance,
drive the plurality of light sources according to the set operating condition, and
estimate a concentration of an analyte component based on a plurality of light quantities detected from skin by the at least one detector.

2. The apparatus of claim 1, wherein the first light source of the plurality of light sources has a central wavelength in a first range from about 350 nm to about 450 nm or in a second range from about 500 nm to about 600 nm.

3. The apparatus of claim 1, wherein the processor is further configured to:
set the sensor to a first operating condition,
drive the first light source based on the first operating condition,
determine the first reflectance based on a first light quantity detected from the skin by the at least one detector, and
based on the determined first reflectance being less than a first threshold value, change the operating condition to a second operating condition.

4. The apparatus of claim 3, wherein the processor is further configured to determine the first reflectance based on an initial light quantity and the first light quantity, and
wherein the initial light quantity is detected from a standard reflector using the first light source based on the first operating condition.

5. The apparatus of claim 4, wherein the processor is further configured to determine, as the first reflectance, a result obtained by dividing the first light quantity by the initial light quantity.

6. The apparatus of claim 3, wherein the processor is further configured to, based on the first reflectance being greater than or equal to the first threshold value:
drive a second light source of the plurality of light sources, and
determine a second reflectance based on a second light quantity detected from the skin by the at least one detector, and
wherein the processor is further configured to, based on the determined second reflectance being greater than a second threshold value, change the operating condition to a third operating condition.

7. The apparatus of claim 6, wherein the second light source has a central wavelength ranging from about 450 nm to about 500 nm.

8. The apparatus of claim 1, wherein the processor is further configured to:
obtain a skin spectrum based on the plurality of light quantities,
determine absorbance based on the obtained skin spectrum, and
estimate the concentration of the analyte component based on the determined absorbance.

9. The apparatus of claim 1, wherein the processor is further configured to, based on a predetermined condition being satisfied, determine an operating condition of the sensor.

10. The apparatus of claim 1, wherein the operating condition of the sensor comprises a drive current for driving the plurality of light sources.

11. The apparatus of claim 1, wherein the processor is further configured to:
set the sensor to a first operating condition,
drive the first light source based on the first operating condition,
determine a plurality of reflectances based on the plurality of light quantities detected from the skin by the at least one detector, and
guide a user to change a position of the sensor based on the determined plurality of reflectances.

12. The apparatus of claim 1, wherein the analyte component comprises at least one of skin carotenoid, blood carotenoid, glucose, urea, lactate, triglyceride, total protein, cholesterol, and ethanol.

13. A method of estimating a concentration of a component, the method comprising:
determining a first reflectance of skin using a first light source of a plurality of light sources, the plurality of light sources being included in a sensor and having different central wavelengths,
setting an operating condition of the sensor based on the determined first reflectance;
driving the plurality of light sources based on the set operating condition; and
estimating a concentration of an analyte component based on a plurality of light quantities detected from the skin by at least one detector included in the sensor.

14. The method of claim 13, wherein the first light source has a central wavelength in a first range from about 350 nm to about 450 nm or in a second range from about 500 nm to about 600 nm.

15. The method of claim 13, wherein the setting of the operating condition of the sensor comprises:
setting the sensor to a first operating condition;
driving the first light source based on the first operating condition;
determining the first reflectance based on a first light quantity detected from the skin by the at least one detector; and
based on the determined first reflectance being less than a first threshold value, changing the operating condition to a second operating condition.

16. The method of claim 15, wherein the determining of the first reflectance comprises:
determining the first reflectance based on an initial light quantity and the first light quantity, and
wherein the initial light quantity is detected from a standard reflector using the first light source based on the first operating condition.

17. The method of claim 15, wherein the setting of the operating condition of the sensor further comprises:
based on the first reflectance being greater than or equal to the first threshold value, driving a second light source of the plurality of light sources;
determining a second reflectance based on a second light quantity detected from the skin by the at least one detector; and
based on the determined second reflectance being greater than a second threshold value, changing the operating condition to a third operating condition.

18. The method of claim 13, wherein the estimating of the concentration of the analyte component comprises:
obtaining a skin spectrum based on the plurality of light quantities;
determining absorbance using the obtained skin spectrum; and
estimating the concentration of the analyte component based on the determined absorbance.

19. A wearable device comprising:
a main body worn on a body of a user;
a sensor comprising:
a plurality of light sources having different central wavelengths, and
a detector configured to detect light from skin of the body of the user; and
a processor configured to:
drive the plurality of light sources based on an operating condition of the sensor,
estimate a concentration of an antioxidant component based on light detected by the detector, and
based on a predetermined condition being satisfied:
determine whether there is a change in color of the skin using a light source of the plurality of light sources having a predetermined central wavelength, and
set an operating condition of the sensor based on the determination of whether there is a change in the color of the skin.

20. The wearable device of claim 19, wherein the predetermined condition is set in advance based on at least one of:
a period of time when the main body is not worn on the body of the user before the antioxidant component is estimated,
a period of time during a day when the main body is not worn on the body of the user, and
a season.
